(19) United States
(12) Patent Application Publication
(10) Pub. No.: US 2015/0202243 A1
(43) Pub. Date: Jul. 23, 2015

(54) HDC Activation Inhibitor, HDC Activation Inhibition Composition, Antipruritic Agent, and Antipruritic Agent Composition

(71) Applicant: HOYU CO., LTD., Aichi (JP)

(72) Inventors: Yoshihiro Inami, Aichi (JP); Kota Hatano, Aichi (JP)

(73) Assignee: HOYU CO., LTD., NAGOYA-SHI, AICHI (JP)

(21) Appl. No.: 14/388,528

(22) PCT Filed: Mar. 27, 2013
(86) PCT No.: PCT/JP2013/059073

§ 371 Date: Sep. 26, 2014

(30) Foreign Application Priority Data
Mar. 28, 2012 (JP) 2012-074733

(51) Int. Cl. A61K 36/736; A61K 31/353; A61K 8/49; A61K 31/7048; A61K 8/60; A61K 31/7024; A61K 31/05; A61K 8/34; A61K 8/97; A61K 36/69; A61K 36/076; A61K 36/53; A61K 36/284; A61K 36/49; A61Q 19/00

(52) U.S. Cl. CPC A61K 36/736; A61Q 19/005; A61Q 19/008; A61K 31/353; A61K 8/498; A61K 31/7048; A61K 8/602; A61K 31/7024; A61K 31/05; A61K 8/347; A61K 8/97; A61K 36/69; A61K 36/076; A61K 36/53; A61K 36/284; A61K 36/49; A61K 2800/75

(57) ABSTRACT

Provided are an HDC activation inhibitor and an antipruritic agent which are effective in animals against the itch caused by a itch generation mechanism different from the well-known one.

An HDC activation inhibitor or antipruritic agent which is at least one member selected from (1) a particular flavonoid, (2) a tannin, (3) chlorogenic acid, (4) a particular stilbenoid, or a glucoside or pharmaceutically acceptable derivative of any of (1) to (4) given above, or (5) a particular crude drug extract, (6) walnut polyphenol.

### FIELD

The present invention relates to an HDC activation inhibitor, an HDC activation inhibition composition, an antipruritic agent, and an antipruritic agent composition. More particularly, this invention relates to a novel HDC activation inhibitor that effectively inhibits the induction of HDC (L-histidine decarboxylase) activation in a newly discovered mechanism for the generation of itch, and a novel HDC activation inhibition composition comprising the same inhibitor, as well as a novel antipruritic agent that exhibits its antipruritic effect by blocking this itch generation mechanism, and a novel antipruritic agent composition comprising the same agent.

### BACKGROUND

Itch, a sensation that is exclusively felt on the skin and mucosa, is so unpleasant that quality of life (QOL) is impaired. Also, itch creates a scratch reflex, thereby causing deterioration of skin symptoms, such as development of rash, through the so-called itch-scratch cycle. The “itch-scratch cycle” refers to the following vicious cycle. Scratching an itching spot damages the skin, sometimes leading to skin inflammation. When inflammation is triggered, itch-inducing factors such as inflammatory cytokines are released from the inflamed site and its vicinity, thereby causing one to feel itch again. Then, the skin is scratched again so that skin symptoms get worse.

For these reasons, itch has been reported as a chief complaint by many individuals with skin diseases, and many antipruritic agents such as chlorpheniramine and diphenhydramine have conventionally been developed, but these agents do not necessarily provide satisfactory antipruritic effects. Underlying this is that no objective and adequate method for evaluating antipruritics has been provided yet. This has something to do with the facts that there are diverse causes for the development of itch and that itch mechanisms remain unclear in many respects.

In the clinical setting, various itch evaluation methods are adopted, such as VAS (Visual Analogue Scale) by which patients are asked to complain of what percentage the intensity of the itch they are experiencing is, relative to that of the most intense itch possible. However, evaluations based on reports given by patients themselves are influenced by their subjectivity, and depend on various factors including their health conditions and mood at the time of evaluation; so it is difficult to directly compare the intensities of itches reported by different individuals.

Under these circumstances, there is a widely known method for evaluating itch through experimentation—a scratch test using as an index the scratch reflex associated with itching in laboratory animals treated with an antipruritic agent or substance, as typically disclosed in Patent Document 1 noted below. In this test, evaluation is made by video-recording the behavior of the animals in an unattended environment and reviewing the recorded video to count the number of scratches; thus, this is an objective evaluation not influenced by human subjectivity. However, in the evaluation method using laboratory animals, the responses exhibited by animals may vary with the type or phyletic line of the animals to be used. In addition, the method disclosed in Patent Document 1 involves only observation of the scratch reflex of animals but not analysis of the “itch generation mechanism” detailed below.

Also known is a test method using cells. For example, the test method disclosed in Patent Document 2 noted below involves the steps of bringing cultured cells capable of expressing PAR2 (protease-activated receptor 2) into contact with a test substance, and measuring the amount of PAR2 expressed. “PAR2” refers to a receptor belonging to the family of G-protein coupled seven transmembrane receptors which are activated by proteases such as trypsin and tryptase. However, such cell culture tests have some problems as mentioned below. First, fat-soluble components which are insoluble in cell culture media are difficult to evaluate. Also, since cell culture media have buffer capacity, it is difficult to convey the influences of pH, etc. to cells. Further, in a cell culture test using mast cells, a test substance having surface activity acts on the cell membrane of mast cells, causing degranulation from the mast cells due to its surface activity.

Meanwhile, there are proposed L-histidine decarboxylase inhibitors comprising an HDC (L-histidine decarboxylase) enzymatic activity inhibiting substance as an active component, as typically disclosed in Patent Documents 3-6 noted below. However, the HDC activity inhibiting substances of these inhibitors inhibit the activity of the HDC that has been present in an activated form in particular cells. Further, the evaluations of these HDC activity inhibiting substances were not made using epidermal samples.

In other words, according to these patent documents, the evaluations of the HDC enzymatic activity inhibiting substances were made by the following procedure: as a preliminary step, the stomachs were dissected from mice and homogenized to obtain suspensions, and then the HDCs present in the suspensions were evaluated in vitro for their enzymatic activity based on the amount of histamine produced. In the stomach, there are ECL (enterochromaffin-like) cells, which contain activated HDC intracellularly and are capable of storing intracellular granules of histamine produced intracellularly. The enzymatic activity of this activated HDC is evaluated by the amount of histamine produced when the substrate L-histidine is added.

### CITAITON LIST

### Patent Documents

Patent Document 1: Japanese Patent No. JP 4437251
Patent Document 2: Japanese Patent Application Publication No. JP 2004-170323
Patent Document 3: Japanese Patent Application Publication No. JP H08-217674
Patent Document 4: Japanese Patent Application Publication No. JP H09-110857
Patent Document 5: Japanese Patent Application Publication No. JP H10-059956
Patent Document 6: Japanese Patent Application Publication No. JP 2006-176480

### DETAILED DESCRIPTION

### Technical Problem

Generally speaking, it is well known that histamine is involved in itch, and that histamine is produced from L-histidine by HDC (L-histidine decarboxylase). It is also well known that activated HDC is present in mast cells.

As regards the mechanism for the generation of itch in humans and animals, the following well-known idea has conventionally been dominant, as suggested directly or indirectly in Patent Documents 3-6: “when a particular irritation is applied to mast cells present in skin dermis, histamine is produced by activated HDC present in the mast cells and released extracellularly by degranulation of the mast cells, and the released histamine is bound to a histamine receptor present in sensory nerves so that the itch signal is transmitted to the central nervous system.”

The above-mentioned methods disclosed in Patent Documents 3-6 are based on this idea; therefore, the evaluations conducted in these methods focus attention on mast cells, more specifically on the inhibition of the activity of HDC present in an activated form in mast cells. Using, as antipruritic agents, the HDC activity inhibitors evaluated and screened in this manner is not denied absolutely and can be presumed to be more or less effective against itching.

However, the study made by the present inventors revealed the presence of a new itch, which cannot be explained by the itch generation mechanism caused by degranulation from mast cells. Therefore, the antipruritic agents screened by conventional evaluation methods predicated on the known itch generation mechanism may not take effect against such a newly discovered itch. In addition, evaluation of agents effective against the new itch cannot be done properly.

Thus, an object to be achieved by the present invention is to provide, based on the clarification of the new itch generation mechanism which cannot be explained by the conventionally known mechanism described above, new antipruritic substances screened by an evaluation method focusing on the newly clarified generation mechanism.

In the course of pursuing a means for achieving the above-mentioned object, the present inventors obtained the following finding about the conventionally unknown itch generation mechanism: particular irritant substances act to induce the activation of inactivated HDC (HDC precursor) present in skin epidermal keratinocytes and, as a result, the keratinocytes produce (generate and extracellularly release) histamine, which causes itching of the skin. The present invention is based on this finding.

### Technical Solution

(Constitution of the First Invention)

The constitution of the first invention for achieving the above-mentioned object is:

an HDC activation inhibitor which is at least one member selected from (1) to (6) mentioned below:

(1) apigenin, luteolin, diosmetin, genkwanin, poncirin, eriodictyol, sterubin, or prunetin as a flavonoid, or a glucoside or pharmaceutically acceptable derivative thereof;

(2) a tannin, or a glucoside or pharmaceutically acceptable derivative thereof;

(3) chlorogenic acid, or a glucoside or pharmaceutically acceptable derivative thereof;

(4) a stilbenoid, including at least resveratrol, or a glucoside or pharmaceutically acceptable derivative thereof;

(5) a Prunus jamasakura bark extract, a Polygala root extract, a Hoelen extract, a Glechoma hederacea extract, or an Atractylodis lanceae rhizoma extract as a crude drug extract;

(6) walnut polyphenol.

For the purpose of the present specification, the expression “HDC activation inhibitor (inhibiting)” as used in relation to a substance or agent refers to a “substance or agent for inhibiting the activation of inactivated HDC present in epidermal keratinocytes”. On the other hand, the expression “HDC activity inhibitor (inhibiting)” as used in relation to a substance or agent refers to a “substance or agent for inhibiting the enzymatic activity of activated HDC present in dermal mast cells”. Hence, the expressions “HDC activation inhibitor (inhibiting)” and “HDC activity inhibitor (inhibiting)” are clearly distinguished from each other in the present invention.

(Constitution of the Second Invention)

The constitution of the second invention for achieving the above-mentioned object is:

an HDC activation inhibition composition comprising the HDC activation inhibitor as set forth in the first invention.

(Constitution of the Third Invention)

The constitution of the third invention for achieving the above-mentioned object is:

the HDC activation inhibition composition as set forth in the second invention, wherein the HDC activation inhibition composition is a pharmaceutical product, a quasi-drug product, or a cosmetic product.

(Constitution of the Fourth Invention)

The constitution of the fourth invention for achieving the above-mentioned object is:

the HDC activation inhibition composition as set forth in the second or third invention, wherein the HDC activation inhibition composition is a skin preparation for external use.

(Constitution of the Fifth Invention)

The constitution of the fifth invention for achieving the above-mentioned object is:

an antipruritic agent which is at least one member selected from (1) to (6) mentioned below:

(1) apigenin, luteolin, diosmetin, genkwanin, poncirin, eriodictyol, sterubin, or prunetin as a flavonoid, or a glucoside or pharmaceutically acceptable derivative thereof;

(2) a tannin, or a glucoside or pharmaceutically acceptable derivative thereof;

(3) chlorogenic acid, or a glucoside or pharmaceutically acceptable derivative thereof;

(4) a stilbenoid, including at least resveratrol, or a glucoside or pharmaceutically acceptable derivative thereof;

(5) a Prunus jamasakura bark extract, a Polygala root extract, a Hoelen extract, a Glechoma hederacea extract, or an Atractylodis lanceae rhizoma extract as a crude drug extract;

(6) walnut polyphenol.

(Constitution of the Sixth Invention)

The constitution of the sixth invention for achieving the above-mentioned object is:

an antipruritic agent composition comprising the antipruritic agent as set forth in the fifth invention.

(Constitution of the seventh invention)

The constitution of the seventh invention for achieving the above-mentioned object is:

the antipruritic agent composition as set forth in the sixth invention, wherein the antipruritic agent composition is a pharmaceutical product, a quasi-drug product, or a cosmetic product.

(Constitution of the eighth invention)

The constitution of the eighth invention for achieving the above-mentioned object is:

the antipruritic agent composition as set forth in the sixth or seventh invention, wherein the antipruritic agent composition is a skin preparation for external use.

### Advantageous Effects

The study made by the present inventors found the presence of a conventionally unknown itch generation mechanism, in which a particular irritation induces the activation of HDC in epidermal keratinocytes, resulting in production of histamine in the keratinocytes and occurrence of itching of the skin due to the release of histamine. Further, the animal test conducted by the inventors confirmed scratching behavior induced by the application of a particular irritant substance, and found that the scratching behavior is a response mediated by the histamine produced by keratinocytes.

The HDC activation inhibitors listed as (1) to (6) in the first invention display a significant effect not achievable by conventional antipruritics: more specifically, in animals, particularly mammals including humans and non-human mammals, the inventive inhibitors can inhibit activation of inactivated HDC present in skin epidermal keratinocytes induced by particular irritant substances, as mentioned above. Also, the antipruritic agents listed as (1) to (6) in the sixth invention display a significant effect not achievable by conventional antipruritics: more specifically, as a result of inhibiting the activation of inactivated HDC as mentioned above, the inventive antipruritic agents suppress or prevent the itch generated by this itch generation mechanism. Accordingly, the first and sixth inventions provide HDC activation inhibitors and antipruritic agents that can take effect against the itch newly discovered by the present inventors.

Since itch is a sensation produced in the surface layer of the skin, it is desirable to apply an irritation to the skin surface layer in an experiment for investigating itch. Such an irritation acts on keratinocytes present in the skin's epidermis earlier than on mast cells present in the skin's dermis; thus, it is presumable that the irritation will exert a stronger action on keratinocytes than on mast cells in the dermal layer. Therefore, the HDC activation inhibitor and antipruritic agent of the present invention may be more advantageous than antipruritic substances screened by conventional evaluation methods. In addition, antipruritic substances for itching of the skin are often used in the form of a skin preparation for external use which is applied onto the skin to thereby suppress itching locally. When a preparation for external use is applied onto the skin, the preparation is absorbed from the skin's outermost layer, stratum corneum, to first act on the epidermis. Since 90% or more of cells constituting the epidermis are keratinocytes, it is conceivable that the inventive HDC activation inhibitor and antipruritic agent are particularly advantageous as antipruritic substances that can be used in the form of a skin preparation for external use.

The HDC activation inhibition composition according to the second invention comprises the HDC activation inhibitor as set forth in the first invention; thus, there is provided an HDC activation inhibition composition that takes effect against the itch newly discovered by the present inventors. Also, the antipruritic agent composition according to the seventh invention comprises the antipruritic agent as set forth in the sixth invention; thus, there is provided an antipruritic agent composition that takes effect against the itch newly discovered by the inventors.

As set forth in the third or seventh invention, the HDC activation inhibition composition or the antipruritic agent composition can be used as a pharmaceutical product, a quasi-drug product, or a cosmetic product.

As set forth in the fourth or eighth invention, the HDC activation inhibition composition or the antipruritic agent composition can be particularly preferably used as a skin preparation for external use to suppress itching of the skin.

### BRIEF DESCRIPTION OF THE DRAWINGS

FIG. 1 shows a working example of the method for evaluating the antipruritic effect of a test substance according to the present invention.

FIG. 2 shows the HDC activation inhibiting effects (antipruritic effects) achieved by test substances in an evaluation using a three-dimensional human epidermis model, in terms of percent HDC activation.

FIG. 3 shows the HDC activation inhibiting effects (antipruritic effects) achieved by comparative test substances in an evaluation using a three-dimensional human epidermis model, in terms of percent HDC activation.

FIG. 4 shows a comparison between the solvent control group and the test substance groups in terms of the number of scratches caused by sodium laurate-induced itching.

### REFERENCE SIGNS LIST

1 Vessel
2 Culture cup
3 Membrane filter
4 Assay medium
5 Stratum corneum
6 Laminar portion
7 Tool for dropwise addition
8 Evaluation solution

### DESCRIPTION OF EMBODIMENTS

Next, embodiments of the present invention will be described, including the best mode thereof.

[Method for Evaluating the HDC Activation Inhibitor or Antipruritic Agent]

First described is the method for evaluating the HDC activation inhibitor or antipruritic agent according to the present invention. This method, which focuses attention on the activation of HDC induced in epidermal keratinocytes of animals, particularly mammals including humans and non-human mammals, by the action of a particular irritant substance, evaluates the HDC activation inhibiting effect or antipruritic effect of test substances on the basis of their inhibitory effect on this activation. The HDC activation inhibitor and antipruritic agent according to this invention is screened by this evaluation method.

To be specific, in this evaluation method, the processes mentioned below in (1) and (2), for example, are performed on epidermal keratinocytes of mammals simultaneously or in tandem, whereby the HDC activation inhibiting effect or antipruritic effect of test substances can be evaluated using the value of percent HDC activation in the keratinocytes as an index:

(1) an activation inducing process in which epidermal keratinocytes of a mammal are irritated to induce the activation of HDC; and

(2) an activation inhibiting process in which a test substance for evaluation is exposed to the epidermal keratinocytes of the mammal or administered to the mammal.

The “percent HDC activation” mentioned above refers to a value expressed as the result of comparing index al which represents the HDC enzymatic activity observed in the case where both processes (1) and (2) above are performed, with index a2 which represents the HDC enzymatic activity observed in the case where substantially only process (1) is performed (control test). The percent HDC activation serves as an effective index for HDC activation inhibiting effect and consequently as an index for antipruritic effect. Here, the mode of comparison between indexes a1 and a2 is not limited, and the percent HDC activation can be expressed, for example, as a value indicating the proportion of a1/a2 or as the percentage of a1/a2 (%). The nature of indexes a1 and a2 for enzymatic activity is also not limited, and the percent HDC activation can be, for example, a parameter indicating the quantitative ratio of activated HDC to inactivated HDC in keratinocytes. Since activated HDC has a molecular mass of about 53 kDa, and inactivated HDC (HDC precursor) about 74 kDa, the quantitative ratio of the two HDCs can be calculated by, for example, western blotting. The “the case where substantially only process (1) is performed (control test)” as mentioned above is exemplified by the case where process (1), but not process (2), is performed, or the case where not only process (1) is performed but also process (2) is carried out using a solvent (e.g., water) alone instead of a solution of a test substance.

Further, the activation inducing means for irritating keratinocytes to induce the activation of HDC is not limited and, for example, a surfactant can be preferably used. Surfactants, which are used not only as skin cleansing agents such as shampoos, body soaps, hand soaps and facial cleansers but also as detergents for washing clothes and dishes, have a basic function of cleansing the skin, but may cause dryness, roughness, itching and other symptoms of the skin. Among such surfactants, sodium laurate and other anionic surfactants are commonly used because of their excellent detergency and thus are frequently contacted with the skin. Some anionic surfactants are irritant to the skin of humans and animals.

The value of percent HDC activation that should be used as a criterion for screening the HDC activation inhibitor or antipruritic agent according to the above-mentioned evaluation method should be decided as appropriate depending on the effect required of the HDC activation inhibitor or antipruritic agent, and is difficult to define uniformly. One criterion that can be mentioned as an example is that “the value of percent HDC activation be 75% or less, particularly preferably 60% or less”.

[HDC Activation Inhibitor and Antipruritic Agent]

The HDC activation inhibitor or antipruritic agent is, in a broad sense, composed of at least one member selected from (1) to (6) mentioned below:

(1) a flavonoid, or a derivative or glucoside thereof;

(2) a tannin, or a derivative or glucoside thereof;

(3) chlorogenic acid, or a derivative or glucoside thereof;

(4) a stilbenoid, or a derivative or glucoside thereof;

(5) a crude drug extract;

(6) walnut polyphenol.

(Flavonoids)

Flavonoids are a class of polyphenols and are generally recognized as a generic name for secondary plant metabolites derived from chalcones formed by polymerizing coumaric acid CoA and malonoyl-CoA. Examples of flavonoids include anthocyanins, flavanes, flavanones, flavones, flavonols, dihydroflavonols, chalcones, aurones, isoflavonoids, and neoflavonoids. Plant extracts containing such substances can be exemplified by Yerba santa leaf extracts.

The HDC activation inhibitor or antipruritic agent according to the present invention is specifically composed of at least one member selected from (1) to (6) mentioned below. No report has been seen or heard on the effects of the below-mentioned substances as conventional antipruritics, let alone as the HDC activation inhibitor or antipruritic agent according to this invention.

(1) Apigenin, luteolin, diosmetin, genkwanin, poncirin, eriodictyol, sterubin, or prunetin as a flavonoid, or a glucoside or pharmaceutically acceptable derivative thereof.

(2) A tannin, or a glucoside or pharmaceutically acceptable derivative thereof.

(3) Chlorogenic acid, or a glucoside or pharmaceutically acceptable derivative thereof.

(4) A stilbenoid, including at least resveratrol, or a glucoside or pharmaceutically acceptable derivative thereof.

(5) a Prunus jamasakura bark extract, a Polygala root extract, a Hoelen extract, a Glechoma hederacea extract, or an Atractylodis lanceae rhizoma extract as a crude drug extract.

(6) Walnut polyphenol.

The “glucoside” refers to products formed by bonding of one or more sugar units such as glucose or galactose to the functional group (e.g. hydroxyl or carboxyl group) of any of the compounds mentioned above in (1) to (4) as long as the unit(s) does(do) not interfere with the HDC activation inhibiting effects or antipruritic effects of the compounds.

The “pharmaceutically acceptable derivative” refers to products formed by bonding of any given compound other than compounds (1) to (4) to the functional group (e.g. hydroxyl or carboxyl group) of any of compounds (1) to (4), or by substitution of a particular carbon atom constituting the ring structure of any of the compounds by any other compound as long as the products are pharmaceutically acceptable. Examples of the pharmaceutically acceptable derivative include various salts, solvates, and esterified products.

Salts can be exemplified by inorganic acid salts (e.g., hydrochloride, sulfate, nitrate, hydrobromate, phosphate), organic acid salts (e.g., carboxylate, oxycarboxylate, organic sulfonate), salts with organic bases (e.g., methylamine, triethylamine, triethanolamine), and salts with inorganic bases (e.g., ammonium salt, alkali metal salts, alkali earth metal salts). Solvates can be exemplified by hydrates, ethanol solvates, methanol solvates, and acetonitrile solvates. Esterified products can be exemplified by carboxylic acid esters, phosphoric acid esters, carbonic acid esters, sulfuric acid esters, nitric acid esters, and thioesters.

Other examples of the derivative include so-called prodrugs. The “prodrugs” means metabolic precursors that can be converted into the compounds of the present invention under physiological conditions.

(Tannin)

For the purpose of the present invention, the tannin refers to an astringent component such as persimmon tannin or chestnut tannin, and also is a generic name for polyphenol compounds contained in the leaves and other parts of a wide variety of plants. Representative tannins include plant tannins extracted from gall or nutgall, persimmon tannin, chestnut skin tannin, tamarind tannin, and mimosa tannin. Other exemplary tannins include so-called hydrolysable tannins (pyrogallol tannins) and condensed tannins (catechol tannins). Specific examples of tannins include tannin acid, and hydrolysable tannins contained in cloves and the like, with tannin acid being more preferred.

(Chlorogenic Acid)

Chlorogenic acid, which is also called 5-caffeoylquinic acid, has a structure in which the carboxyl group of caffeic acid is dehydration-condensed with the hydroxy group at position 5 of quinic acid. Chlorogenic acid is obtained not only from coffee beans but also from seeds and leaves of many other dicotyledonous plants. Examples of dicotyledonous plants containing chlorogenic acid include cherry leaves.

(Stilbenoid)

The stilbenoid is a compound having a structure in which a compound formed by bonding of 3 units of malonoyl-CoA to p-hydroxycinnamic acid CoA is ring-closed. The stilbenoid is exemplified not only by stilbenes (e.g., resveratrol and rhaponticin), phyllodulcin, oligostilbenes, and polystilbenes, but also by resveratrol oligomers, such as the resveratrol dimers c-viniferin and gnetin C, or the resveratrol dimer glucosides gnemonosides A, C and D, or the resveratrol trimer α-viniferin, or the resveratrol tetramer vaticanol C. The preferred systematic name of resveratrol is 3,5,4′-trihydroxy-trans-stilbene.

(Walnut Polyphenol)

The walnut polyphenol is a hydrolysable polyphenol which is a component contained in the seed coat (pellicle) of walnuts.

[HDC Activation Inhibition Composition and Antipruritic Agent Composition]

(Active Component, and Purpose of Use, Dosage Form, etc.)

The HDC activation inhibition composition or antipruritic agent composition according to the present invention comprises the above-mentioned HDC activation inhibitor or antipruritic agent as an active component. The content of the HDC activation inhibitor in the HDC activation inhibition composition, or the content of the antipruritic agent in the antipruritic agent composition is not limited, and these contents can each be in the range of, for example, 0.1 to 50% by mass. If these contents are less than 0.1% by mass, a sufficiently satisfactory effect may not be obtained, and if these contents exceed 50% by mass, there may occur a problem with solubility or the like.

The HDC activation inhibition composition or antipruritic agent composition of the present invention can be used, as a pharmaceutical product, a quasi-drug product, or a cosmetic product which have various purposes of use, to treat or prevent various symptoms accompanied by itching. One particularly preferred example is a skin preparation for external use, and other preferred examples include a medicine for internal use and an injectable drug.

Examples of purposes of use of the skin preparation for external use include: dermatitis therapeutic agents or antipruritic agents for treating itch and inflammation caused by various troubles including dry skin/xeroderma, skin keratosis, mild atopic dermatitis, seborrheic dermatitis, papule, erythema, eczema, rash, dry pruritus, seborrheic pruritus, urticaria, insect bites, chilblains, and sudamen; therapeutic agents for treating or preventing the worsening of cuts, abrasions, shoe sores, scratches, purulent wounds, cracks, chaps and the like, or infectious skin disease therapeutic agents for treating athlete's foot, tinea, acne, and the like; pharmaceutical products such as keratin softeners for treating rough hands and fingers, keratosis on elbows, knees, heels, ankles and the like, and dry scaly skin; quasi-drug products for use as in prevention of rough hands, skin and lips, and chilblains, cracks, chaps, acne, rash and the like; and skin cosmetic products used as for suppression or prevention of itch.

The HDC activation inhibition composition or antipruritic agent composition of the present invention can be prepared into various dosage forms; for example, solid medicines including stick form, ointments, liquid medicines including lotion, emulsion, or aerosol form, foams, gels, creams, and patches including pack form. In particular, ointments, liquid medicines, gels, and creams are preferred.

The method for preparing the inventive HDC activation inhibition composition or antipruritic agent composition into such various dosage forms is not particularly limited, and the inventive composition can be prepared into such dosage forms according to a conventional technique by selecting and mixing various components as appropriate. The dose and usage of the inventive HDC activation inhibition composition or antipruritic agent composition are also not particularly limited, and the inventive composition can be commonly used in a proper dose a few times a day.

(Other Components)

As long as the effects of the present invention are not impaired, one or more conventional antipruritic agents, or in other words one or more known antipruritic agents screened as substances inhibiting the enzymatic activity of activated HDC present in mast cells, can be incorporated in the inventive HDC activation inhibition composition or antipruritic agent composition.

Examples of such known antipruritic agents include chlorpheniramine, diphenhydramine, lidocaine, dibucaine, ethyl aminobenzoate, cyproheptadine, diphenylpyraline, triprolidine, promethazine, homochlorcyclizine, ammonia, capsaicin, nonanoic acid vanillylamide, salicylic acid, methyl salicylate, glycol salicylate, alimemazine, clemastine, mequitazine, dexamethasone, betamethasone, dexamethasone valerate acetate, prednisolone valerate acetate, hydrocortisone butyrate, prednisolone acetate, prednisolone, hydrocortisone acetate, hydrocortisone, cortisone acetate, clobetasone butyrate, triamcinolone acetonide, crotamiton, thymol, eugenol, menthol, camphor, hinokitiol, polyoxyethylene lauryl ether, comfrey extracts, perilla extracts, sage extracts, moutan bark extracts, and Tilia miqueliana extracts, as well as pharmacologically (pharmaceutically) or physiologically acceptable salts thereof.

Further, as long as the effects of the present invention are not impaired, the HDC activation inhibition composition or antipruritic agent composition of this invention can contain any one or more of various components that may be included in the fields of pharmaceutical products, quasi-drug products, or cosmetic products. Examples of such components include those listed below in (a) to (g):

(a) anti-inflammatory agents: e.g., glycyrrhizic acid or derivatives thereof such as dipotassium glycyrrhizinate and monoammonium glycyrrhizinate, glycyrrhetic acid or derivatives thereof, allantoin or derivatives thereof, indomethacin, ibuprofen, ibuprofen piconol, bufexamac, butyl flufenamate, bendazac, piroxicam, ketoprofen, felbinac, and salicylic acid derivatives such as methyl salicylate or glycol salicylate;

(b) vitamins: e.g., vitamin A such as retinol, provitamin A such as β-carotene and lycopene, vitamin E such as tocopherol, vitamin B2 such as riboflavin and flavin mononucleotide, vitamin C such as ascorbic acid and dehydroascorbic acid, vitamin D such as ergocalciferol and cholecalciferol, vitamin K such as phylloquinone, vitamin B1 such as γ-orizanol and thiamin, vitamin B6 such as pyridoxine and pyridoxal, vitamin B12 such as cyanocobalamin, folic acids also called pteroylglutamic acid, vitamin B3 such as nicotinic acid and nicotinic acid amide, and pantothenic acids such as coenzyme A;

(c) antimicrobial agents: e.g., isopropylmethylphenol, chlorhexidine hydrochloride, chlorhexidine gluconate, benzalkonium chloride, cetylpyridinium chloride, polyhexamethylene biguanide, triclosan, trichlorocarbanilide, cresol, and piroctone olamine;

(d) antifungal agents: e.g., itraconazole, amorolfine hydrochloride, croconazole hydrochloride, terbinafine hydrochloride, neticonazole hydrochloride, butenafine hydrochloride, clotrimazole, ketoconazole, ciclopiroxolamine, isoconazole nitrate, econazole nitrate, oxiconazole nitrate, sulconazole nitrate, bifonazole, pimaricin, fluconazole, flucytosine, miconazole, and lanoconazole;

(e) humectants: e.g., glycerin, ethylene glycol, propylene glycol, polyethylene glycol, diglycerin trehalose, heparinoids, sodium chondroitin sulfate, collagen, elastin, chitin, chitosan, glycine, asparatic acid, sodium lactate, urea, sodium pyrrolidone carboxylate, ceramide, cholesterol, phospholipids, Chamamila recutita extracts, Aloe (vera) extracts, Hamamelis extracts, rosemary extracts, thyme herb extracts, green tea extracts, and perilla extracts;

(f) whitening agents: e.g., vitamins mentioned above, such as vitamins A, C, E and pantothenic acids; as well as placenta, arbutin, kojic acid, cysteine, phytic acid, iris, almond, Aloe (vera), Ginkgo biloba, oolong tea, rose fruit, Scutellaria root, Coptis japonica, Hypericum erectum, Lamium album, marine algae, Pueraria root, cape jasmine, sophora root, wheat, rice germ, rice bran, Perilla, peony, Cnidium officinale, mulberry bark, glycine max, tea, Japanese angelica root, Carthamus tinctorius, moutan bark, coix seed, and nettle tree;

(g) other components, as exemplified by various astringent agents (e.g., citric acid, zinc sulfate, and marine alga extracts), antioxidant agents (e.g., dibutyl hydroxytoluene, sodium edetate, and sodium sulfite), and anti-wrinkle agents (e.g., acylated glucosamines, kinetin, and hyaluronic acid).

(Pharmaceutical Components)

The HDC activation inhibition composition or antipruritic agent composition of the present invention can further contain a base, a surfactant, a thickening agent, a preservative, a pH adjustor, a stabilizing agent, an irritation reducing agent, an antiseptic agent, a coloring agent, a dispersant, a fragrance, and/or the like, depending on the needs for pharmaceutical production and as long as the effects of this invention are not impaired.

Examples of the base include liquid paraffin, paraffin, vaseline, microcrystalline wax, talc, myristic acid, lauryl alcohol, cetyl alcohol, cetyl octanoate, isopropyl palmitate, dipentaerythritol fatty acid ester, glycerol trimyristate, methyl polysiloxane, crosslinked polyether-modified silicone, and crosslinked alkyl-modified silicone.

Examples of the surfactant include sorbitan fatty acid esters, glycerides, polyglycerides, propylene glycol fatty acid esters, hydrogenated castor oil derivatives, polyoxyethylene sorbitan fatty acid esters, and glycerol alkyl ethers.

Examples of the thickening agent include guar gum, carrageenan, xanthan gum, dextran, methylcellulose, hydroxyethylcellulose, hydroxypropylcellulose, sodium alginate, propylene glycol alginate, polyvinyl alcohol, polyvinylpyrrolidone, polyvinyl methyl ether, carboxyvinyl polymers, sodium polyacrylate, polyethylene glycol, bentonite, and dextrin fatty acid esters.

Examples of the preservative include benzoic acid, sodium benzoate, dehydroacetic acid, butyl parahydroxybenzoate, ethyl parahydroxybenzoate, benzyl parahydroxybenzoate, methyl parahydroxybenzoate, and phenoxyethanol.

Examples of the pH adjustor include inorganic acids (e.g., hydrochloric acid, sulfuric acid, phosphoric acid, polyphosphoric acid, and boric acid), organic acids (e.g., lactic acid, acetic acid, citric acid, tartaric acid, succinic acid, and oxalic acid), gluconolactone, ammonium acetate, inorganic bases (e.g., sodium hydrogencarbonate, sodium carbonate, potassium hydroxide, and sodium hydroxide), and organic bases (e.g., monoethanolamine, triethanolamine, diisopropanolamine, and lysine).

### EXAMPLES

Next, working examples of the present invention will be described. The technical scope of this invention is not limited by the working examples described below.

### Example 1

### Evaluation of Test Substances using Percent HDC Activation

Test substances were evaluated for their HDC activation inhibiting effect (their antipruritic effect) using a three-dimensionally cultured human skin model.

(Three-Dimensionally Cultured Human Skin)

The “three-dimensionally cultured human skin” (hereinafter referred simply to as “cultured skin”) refers to a cultured skin (cultured epidermis) obtained by culturing and stratifying normal human epidermal cells. The cultured skin has a morphologically similar structure to human epidermis (i.e., has stratum corneum, stratum granulosum, stratum spinosum, and stratum basale) and thus is useful as an alternative material for use in a skin irritancy test using a laboratory animal model. Therefore, this cultured skin is a “three-dimensionally cultured human epidermis”, to be exact, and contains no dermal layer of the skin.

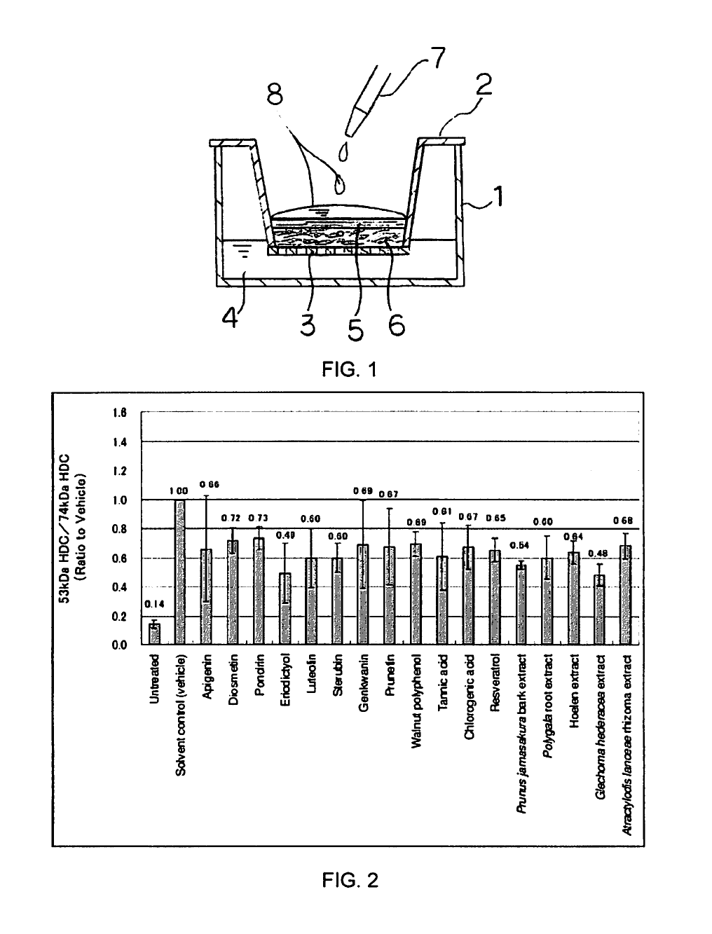

In this Example, the evaluation was made by the apparatus shown in FIG. 1 using the three-dimensionally cultured human skin model “LabCyte EPI-MODEL” produced by Japan Tissue Engineering Co., Ltd. The cultured skin samples of the same size (with the same stratum corneum surface area) were used in the evaluations of the test substance, control and other groups. The apparatus shown in FIG. 1, and the evaluation method using said apparatus are described below.

(Evaluation Apparatus and Method)

In the apparatus shown in FIG. 1, a vessel 1 which is open at its upper side was charged in advance with an assay medium 4 to such a level that the portion of a culture cup 2, which includes a membrane filter 3, was immersed. Next, a cultured skin having stratum corneum 5 and a laminar portion 6 (consisting of stratum granulosum, stratum spinosum, and stratum basale) was placed on the membrane filter 3 in the culture cup 2, and then the culture cup 2 was put in the vessel 1.

In this setting, the cultured skin was first preincubated using an incubator at 37° C. in 5% CO₂ for 1 to 2 hours to allow the cultured skin to be stabilized. Next, the activation inducing process was performed. To be specific, 100 μL of a 1% (w/v) aqueous solution of sodium laurate (“SL”), an activation inducing substance (an irritant substance for inducing the activation of HDC), was added dropwise onto the stratum corneum 5 of the cultured skin using an appropriate tool for dropwise addition 7, and the cultured skin was irritated for 1 minute to trigger the induction of HDC activation. Thereafter, the 1% SL solution was removed, and the cultured skin was washed with distilled water three times and then postincubated in the incubator at 37° C. in 5% CO₂ for 3 hours.

Next, 200 μL of an evaluation solution 8, a solution of each of the below-mentioned test substances, was added dropwise onto the stratum corneum 5 using a new tool for dropwise addition which was different from the one used in the previous process, to allow the cultured skin to be exposed to the evaluation solution (activation inhibiting process), and further the cultured skin was postincubated for 2 hours.

The test substances used were: apigenin, luteolin, diosmetin, genkwanin, poncirin, eriodictyol, sterubin, and prunetin as flavonoids; tannic acid as a tannin; chlorogenic acid; resveratrol as a stilbenoid; a Prunus jamasakura bark extract, a Polygala root extract, a Hoelen extract, a Glechoma hederacea extract, and an Atractylodis lanceae rhizoma extract as crude drug extracts; and walnut polyphenol.

As regards the solutions prepared as above with these test substances, tannic acid and walnut polyphenol were used in the form of a 5% (w/v) solution prepared by dissolution in the solvent water. Regarding the term “(w/v)” as used herein, “w” is the weight in gram (g) and “v” is the volume in milliliter (mL) (the same applies hereunder). The above-mentioned crude drug extracts were used in the form of a 1% (W/V) test substance solution prepared by dissolution in the solvent water. The other test substances listed above were used in the form of a solution prepared by dilution with DMSO to 1×10⁻² M and then with water to 1×10⁻⁶ M (a solution prepared by dissolution in a mixed solvent of water and DMSO (10000:1)).

After the postincubation, the cultured skin was recovered, and proteins were extracted from the cultured skin using the Mammalian Cell Lysis Kit (MCL1) produced by Sigma-Aldrich, Inc. as a solution for protein extraction.

The resulting protein liquid extract was centrifuged to obtain a supernatant as a protein solution. The protein solution was quantified for protein content using the protein quantification kit 2-D Quant Kit produced by GE Healthcare Bioscience Co., Ltd. Then, to a fixed amount of the proteins was added by a sample buffer containing the reducing agent 2-mercaptoethanol, and the mixture was reacted at 95° C. to thereby cleave disulfide bonds in the protein structure. This treatment allows the protein solution to undergo an electrophoresis which reflects the molecular weights of activated HDC (53 kDa) and inactivated HDC (74 kDa).

The thus-treated protein solution was subjected to western blotting. More specifically, the protein solution was subjected to electrophoresis at 200 V for about 100 minutes using electrophoresis gels (NuPAGE 4%-12% Bis-Tris Gels produced by Invitrogen Corporation). As a result, the proteins were separated by molecular weight. Next, the separated proteins were transferred to membranes. The membranes were immunostained to detect HDC and β-actin (as a housekeeping protein). β-actin is a protein that is hard to be affected by irritation, and serves as a correction factor for HDC which is easily affected by irritation with the SL solution and the test substances.

The primary and secondary antibodies used for immunostaining were a rabbit polyclonal antibody against HDC (Progen Biotechnik GmbH, Heiderberg, Germany) and a fluorophore-labeled donkey anti-rabbit IgG (H+L) antibody (Invitrogen Corporation, Carlsbad, Calif., USA), respectively. The bands for the detected HDC were digitized using the imaging software Scion Image (Scion Corporation, Frederick, Md., USA). Scion Image is a software that selects the band(s) for the protein(s) to be digitized, reads the area and color density of the band(s), and detects a value(s) corresponding to the amount(s) of the protein(s) expressed, on the basis of these attributes. The detected values were used to calculate a percent HDC activation (X) according to the equation given below. β-actin was also detected in the same way as HDC. The primary and secondary antibodies used for this detection were an antibody against β-actin (rabbit polyclonal antibody against β-actin (Abcam, Tokyo, Japan)) and the anti-rabbit IgG antibody mentioned above, respectively. Furthermore, a solvent containing no test substance was used as a reference control.

X=[(a1−53e/a1−74e)/(a2−53c/a2−74c)] (Equation)

In this equation, “a1−53e” is a band value (a value obtained by imaging the band using Scion Image) for the activated (53 kDa) HDC present after triggering of the induction of HDC activation with the 1% SL solution, followed by exposure to a test substance solution, and “a1−74e” is a band value for the inactivated (74 kDa) HDC present after triggering of the induction of HDC activation with the 1% SL solution, followed by exposure to a test substance solution. “a2−53c” is a band value for the activated (53 kDa) HDC present after triggering of the induction of HDC activation with the 1% SL solution, followed by exposure to the solvent alone which was used to prepare the test substance solution, and “a2−74c” is a band value for the inactivated (74 kDa) HDC present after triggering of the induction of HDC activation with the 1% SL solution, followed by exposure to the solvent alone which was used to prepare the test substance solution.

FIG. 2 shows the percent HDC activation (X) values calculated for the above-mentioned test substances according to the above-mentioned equation, together with those for the “untreated” and “solvent control” groups. “Untreated” refers to the case where neither the 1% SL solution nor any test substance solution was applied, and “solvent control” refers to the case where the 1% SL solution and distilled water instead of a test substance solution were applied.

Regarding the HDC activation inhibiting effect of the test substances, a possible criterion for judgment is that a test substance be judged to be effective as the HDC activation inhibitor (antipruritic agent) of the present invention if the percent HDC activation of the test substance is 75% or less of that of the solvent control group. According to this judgment criterion, the above-mentioned test substances can be judged to be effective.

### Comparative Example 1

### Evaluation of Comparative Test Substances using Percent HDC Activation

Comparative test substances were evaluated for their HDC activation inhibiting effect (antipruritic effect) using the evaluation apparatus shown in FIG. 1 in which the cultured skin was placed as in the case of Example 1.

To be specific, as in the case of Example 1, the cultured skin was stabilized by preincubation, and the SL solution was added dropwise onto the stratum corneum of the cultured skin to irritate the cultured skin. Thereafter, the SL solution was removed, and the cultured skin was washed and then postincubated. Next, 200 μL of an evaluation solution, a solution of each of the comparative test substances, was added dropwise onto the stratum corneum 5 to allow the cultured skin to be exposed to the evaluation solution, and further the cultured skin was postincubated for 2 hours.

The comparative test substances used were: chrysoeriol and hesperatin as flavonoids; diphenhydramine hydrochloride as a known antihistamic agent; and d-chlorpheniramine maleate. Regarding the solutions of the comparative test substances, these substances were used in the form of a solution prepared by dilution with DMSO to 1×10⁻² M and then with water to 1×10⁻⁶ M (a solution prepared by dissolution in a mixed solvent of water and DMSO (10000:1)).

After the postincubation, as in the case of Example 1, the cultured skin was recovered, proteins were extracted from the cultured skin, and the resulting protein liquid extract was centrifuged to obtain a supernatant as a protein solution. Then, the protein solution was quantified for protein content, and a treatment for cleaving disulfide bonds in the protein structure was carried out to allow the protein solution to undergo an electrophoresis which reflects the molecular weights of activated HDC (53 kDa) and inactivated HDC (74 kDa).

As in the case of Example 1, the thus-treated protein solution was subjected to western blotting to separate the proteins, the separated proteins were transferred to membranes, and the membranes were immunostained to detect HDC. The bands for the detected HDC were digitized using the imaging software Scion Image, and the resulting values were used to calculate a percent HDC activation (X) according to the equation given above.

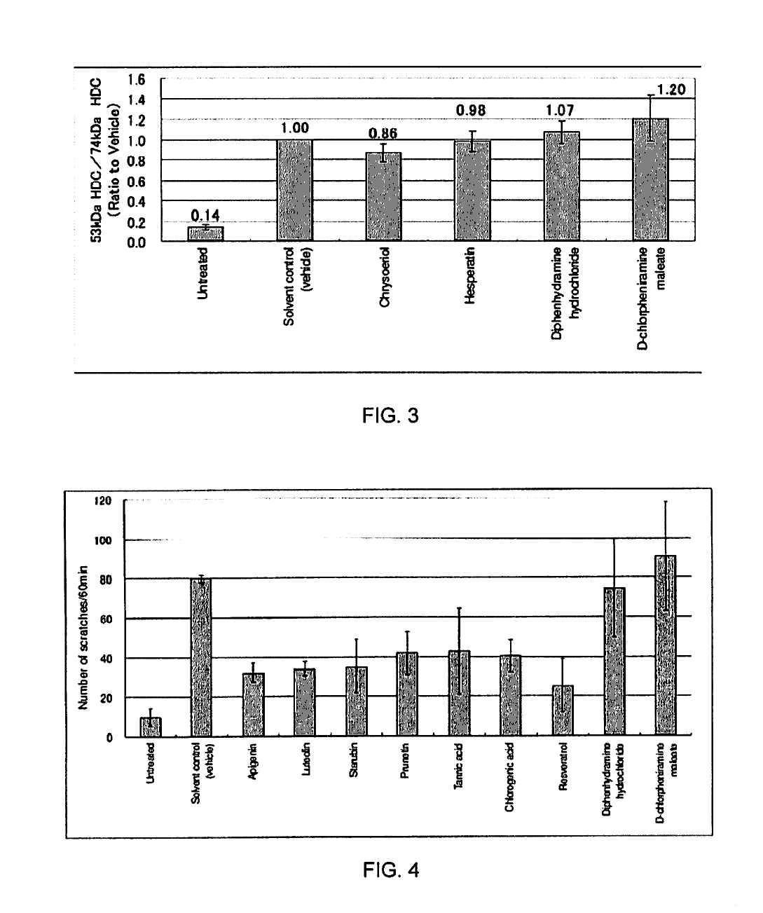

FIG. 3 shows the percent HDC activation (X) values calculated for the comparative test substances according to the above equation, together with those for the “untreated” and “solvent control” groups. Regarding the HDC activation inhibiting effect of the comparative test substances, they all can be judged to be ineffective according to the judgment criterion for effectiveness as described in Example 1, which requires that “the percent HDC activation . . . is 75% or less of that of the solvent control group”.

### Example 2

### Inhibitory Effect Against Scratching Behavior in Mice

Test substances were evaluated for their HDC activation inhibitor (antipruritic agent) effect on the basis of their inhibitory effect on mice's scratching behavior induced by irritation with SL. The test substances used were apigenin, luteolin, sterubin, prunetin, tannic acid, chlorogenic acid, resveratrol, diphenhydramine hydrochloride, and d-chlorpheniramine maleate. Among these substances, diphenhydramine hydrochloride and d-chlorpheniramine maleate were conventionally known as antihistamic agents and served as comparative test substances.

Procedure: the test animals used were male ICR mice aged 7-8 weeks. At least 3 days before the test was conducted, the mice were shaved over a 6 cm² (2×3 cm) area on the rostral back.

The mice were divided into test substance group (n=3 or 4) and solvent control group (n=3). Fifty microliters of a 10% SL aqueous solution was applied to the rostral back of the mice to induce the mice's scratching behavior. Ninety minutes after the application of the SL solution, fifty microliters of a solution prepared with 50% ethanol to give a test substance concentration of 2.5% by mass was applied to the rostral back of the mice of the test substance group, and fifty microliters of 50% ethanol alone was applied to that of the solvent control group. The scratching behavior in the mice was evaluated by the following procedure.

More specifically, the mice treated as above were housed in acrylic cages (26×18×30 cm) divided into 4 sections, one mouse per section, and after the mice were acclimated for at least 30 minutes in an unattended environment, the behavior of the mice was recorded on video for 60 minutes. The recorded video was reviewed to check for the scratching behavior of the mice and visually count the number of a series of scratching behaviors which consist of an animal scratching the rostral back with a posterior leg and putting down the leg. Additionally, in the untreated group (n=4), the mice shaved on the rostral back but not treated with any of a SL solution, a test substance or 50% ethanol were evaluated for their scratching behavior by the same procedure.

The evaluation results for scratching behavior are shown in FIG. 4. This figure shows the mean plus/minus standard deviation of the number of scratches for 60 minutes in each group. According to these evaluation results, the number of scratches for 60 minutes is about 80 to 90 times in the solvent control, diphenhydramine hydrochloride, and d-chlorpheniramine maleate groups, while that number is about 40 times or less in all the test substance groups from apigenin to resveratrol. Regarding the inhibitory effect of the test substances on the number of scratches in mice, a possible criterion for judgment is that a test substance be judged to be effective as the HDC activation inhibitor (antipruritic agent) of the present invention if the number of scratches in the test substance group is 80% or less of that in the solvent control group. According to this judgment criterion, it is not certain whether or not diphenhydramine hydrochloride and d-chlorpheniramine maleate are effective as the HDC activation inhibitor (antipruritic agent) of the present invention, while all of the test substances can be judged to be significantly effective.

In addition, with regard to FIG. 4, the values of the following two variables are listed below for each group: (a) the mean plus/minus standard deviation of the number of scratches for 60 minutes, and (b) the average value for the percentage of the number of scratches for 60 minutes in each group relative to that in the solvent control group which is taken as 100%.

Untreated group: (a) 10±4, (b)—

Solvent control group: (a) 80±2, (b) 100%

Apigenin group: (a) 32±5, (b) 40.5%

Luteolin group: (a) 34±3, (b) 43.0%

Sterubin group: (a) 35±14, (b) 44.4%

Prunetin group: (a) 42±11, (b) 52.7%

Tannic acid group: (a) 43±22, (b) 53.7%

Chlorogenic acid group: (a) 41±8, (b) 50.8%

Resveratrol group: (a) 25±14, (b) 31.7%

Diphenhydramine hydrochloride group: (a) 75±25, (b) 93.8%

D-chlorpheniramine maleate group: (a) 91±28, (b) 113.6%

INDUSTRIAL APPLICABILITY

The present invention provides an HDC activation inhibitor and an antipruritic agent which are effective in animals against the itch caused by a itch generation mechanism different from the well-known one.

1. An HDC activation inhibitor which is at least one member selected from (1) to (6) mentioned below:
(1) apigenin, luteolin, diosmetin, genkwanin, goncirin, eriodictyol, sterubin, or prunetin as a flavonoid, or a glucoside or pharmaceutically acceptable derivative thereof;
(2) tannin, or a glucoside or pharmaceutically acceptable derivative thereof;
(3) chlorogenic acid, or a glucoside or pharmaceutically acceptable derivative thereof;
(4) a stilbenoid, including at least resveratrol, or a glucoside or pharmaceutically acceptable derivative thereof;
(5) Prunus jamasakura bark extracts, Polygala root extracts, Hoelen extracts, Glechoma hederacea extracts, or Atractylodis lanceae rhizoma extracts, all of which are crude drug extracts;
(6) walnut polyphenol.

2. An HDC activation inhibitor composition comprising the HDC activation inhibitor according to claim 1.

3. The HDC activation inhibitor composition according to claim 2, wherein the HDC activation inhibitor composition is a pharmaceutical product, a quasi-drug product, or a cosmetic product.

4. The HDC activation inhibitor composition according to claim 2, wherein the HDC activation inhibitor composition is a skin preparation for external use.

5. An antipruritic agent which is at least one member selected from (1) to (6) mentioned below:
(1) apigenin, luteolin, diosmetin, genkwanin, goncirin, eriodictyol, sterubin, or prunetin as a flavonoid, or a glucoside or pharmaceutically acceptable derivative thereof;
(2) tannin, or a glucoside or pharmaceutically acceptable derivative thereof;
(3) chlorogenic acid, or a glucoside or pharmaceutically acceptable derivative thereof;
(4) a stilbenoid, including at least resveratrol, or a glucoside or pharmaceutically acceptable derivative thereof;
(5) Prunus jamasakura bark extracts, Polygala root extracts, Hoelen extracts, Glechoma hederacea extracts, or Atractylodis lanceae rhizoma extracts, all of which are crude drug extracts;
(6) walnut polyphenol.

6. An antipruritic agent composition, comprising the antipruritic agent according to claim 5.

7. The antipruritic agent composition according to claim 6, wherein the antipruritic agent composition is a pharmaceutical product, a quasi-drug product, or a cosmetic product.

8. The antipruritic agent composition according to claim 6, wherein the antipruritic agent composition is a skin preparation for external use.
